(19) United States
(12) Patent Application Publication
(10) Pub. No.: US 2022/0309137 A1
(43) Pub. Date: Sep. 29, 2022

(54) SECURITY MARKERS

(71) Applicant: CRIME SOLUTIONS LIMITED, Leeds (GB)

(72) Inventor: Paul Maxwell, Leeds (GB)

(21) Appl. No.: 17/309,424

(22) PCT Filed: Nov. 27, 2019
(86) PCT No.: PCT/EP2019/082815

§ 371 Date: May 26, 2021

(30) Foreign Application Priority Data
Nov. 27, 2018 (GB) 1819256.7

(51) Int. Cl. G06F 21/32; C12Q 1/6816

(52) U.S. Cl. CPC G06F 21/32; C12Q 1/6816; C12Q 1/6844

(57) ABSTRACT

A security marker for cooperation with a detector, the detector including a molecular probe, the probe including a predetermined probe nucleotide sequence. The security marker comprises a target oligonucleotide. The target oligonucleotide comprises a pair of primer regions and a marker region located between the primer regions. The marker region comprises a predetermined marker nucleotide sequence which is fully or partially complementary to the probe nucleotide sequence.

### CROSS REFERENCE TO RELATED APPLICATIONS

This is a U.S. national phase application claiming priority to PCT/EP2019/082815, filed Nov. 27, 2019, which claims priority to British application No. 1819256.7, filed Nov. 27, 2018, the entire contents of which are hereby incorporated by reference in their entirety.

### TECHNICAL FIELD

This invention is for a security device that uses DNA codes to create a marker for objects, liquids and people. The invention includes development of the DNA code marker, methods of use for the marker, development of detection for the marker and security devices containing the marker. A database for holding the details of the codes and deployment of security devices is also included in the patent.

### BACKGROUND

Security markers for identifying the ownership of an article are known in the art. Some markers use a specific formulation of different chemical compounds to identify that an object is owned by somebody or was at a specific location.

Security markers comprising the use of nucleic acid molecules are also known in the art, as shown in, for example, WO 94/04918, WO 87/06383 and WO 95/02702. Such security markers may be simply identified using a suitable probe, which may hybridize to the nucleic acid within the marker. The use of polymerase chain reaction (PCR) to isolate and multiply a sample of nucleic acid within a security marker is known, as shown in WO 90/14441.

An advantage of using a nucleic acid molecule as a security marker is that a unique nucleotide sequence can be used for each user of the security marker or for each location in which a security marker is used. Use of oligonucleotides with random nucleic acid sequences is disclosed in U.S. Pat. No. 5,139,812.

The inventors have previously developed an improved security marker (European Patent Application No. 00977665.9, based on PCT/GB00/04419 Security System Crime Solutions Limited).

Such security markers are used as evidential information, whereby they are sprayed upon an intruder upon activation of a suitable actuator. In the latter case, such security markers provide information after the event (proof of presence at the time and location of a crime being committed). Where such a marker is used for the purposes of crime detection it is important that the detection of the marker is as error-free as possible to reduce the possibility of falsely convicting third parties.

European Patent Application No. 00977665.9 discloses a security system based on nucleic acid molecules that were used as unique DNA codes. This design of a single oligonucleotide with binding sites for a primer pair and a uniquely coded central region has a number of disadvantages:

(i) Although the oligonucleotide is unique, less than 1% of its minimum production scale is used. This makes it costly and very inefficient, which reduces the applications it can be used for and restricts its market potential.

(ii) Analysis must be performed within a forensic laboratory, with specialist equipment and knowledge of the sample to be tested.

In this specification, the terms used are to be given their normal meaning as understood by a person skilled in the technical field, unless defined herein.

Abbreviations

PCR=Polymerase Chain Reaction

Tm=melt temperature peak

Tms=melt temperature peaks

### STATEMENTS OF INVENTION

According to a first aspect of the present invention, there is provided a security marker for cooperation with a detector, the detector including a molecular probe, the probe including a predetermined probe nucleotide sequence, the security marker comprising a target oligonucleotide, the target oligonucleotide comprising a pair of primer regions and a marker region located between the primer regions, the marker region comprising a predetermined marker nucleotide sequence which is fully or partially complementary to the probe nucleotide sequence.

Possibly, the marker region is selected from the group containing:
the marker region is fully complementary to the probe nucleotide sequence, the marker nucleotide sequence including no nucleotide mismatches with the probe nucleotide sequence,
the marker region is partially complementary to the probe, the marker nucleotide sequence including a single nucleotide mismatch with the probe nucleotide sequence,
the marker region is partially complementary to the probe, the marker nucleotide sequence including two or more nucleotide mismatches with the probe nucleotide sequence.

Possibly, the security marker includes a plurality of the target oligonucleotides, each of which may be different from the others.

Possibly, the detector includes a plurality of the probes. The security marker may include a corresponding plurality of probe sets of the target oligonucleotides. Each probe set may correspond to a different probe so that the target oligonucleotides of each probe set are fully or partially complementary only to the respective corresponding probe.

Possibly, the security marker includes a plurality of primer region sets. Each primer region set may comprise target oligonucleotides having the same pairs of primer regions. The primer region pairs of the target oligonucleotides of each primer region set may be different to the primer region pairs of the target oligonucleotides of the other set(s).

Possibly, the or each target oligonucleotide comprises a target identity. The target identity may comprise a probe identity relating to the respective complementary probe. The target identity may comprise a primer identity relating to the respective primer region pair. The target identity may comprise a complementarity identity relating to the number of nucleotide mismatches between the respective complementary probe nucleotide sequence and the marker nucleotide sequence.

The security marker may comprise a security code which comprises the target identity or identities of the or all of the target oligonucleotides.

Possibly, the target oligonucleotide(s) comprising the security marker is/are selected from a pool comprising a plurality of the target oligonucleotides. Possibly, each of the target identities of the target oligonucleotides in the pool are different from each other.

Possibly, the or each target oligonucleotide is identified by subjecting the security marker to one PCR reaction for the or each primer pair in the presence of the or all of the probes, possibly then performing a melt curve analysis to generate melting temperature curves for the or each of the primer pairs, and possibly then analysing the curves to generate the security code.

The security marker may include a fluorescent compound, which emits light when exposed to ultra violet light.

According to a second aspect of the present invention, there is provided a detector for cooperation with the security marker defined above, the detector the detector including a molecular probe, the probe including a predetermined probe nucleotide sequence, the security marker comprising a target oligonucleotide, the target oligonucleotide comprising a pair of primer regions and a marker region located between the primer regions, the marker region comprising a predetermined marker nucleotide sequence which is fully or partially complementary to the probe nucleotide sequence.

Possibly, the detector includes a plurality of the molecular probes. Each probe may have a different probe nucleotide sequence. The or each molecular probe may comprise a hybridisation beacon, which may comprise a fluorescent material. Possibly, the or each molecular probe comprises a HyBeacon® probe.

According to a third aspect of the present invention, there is provided a security arrangement including the security marker described above. The arrangement may include a device for applying the security marker to an item, and possibly a database which holds data relating to the security marker.

The security arrangement may include the detector described above, for detecting the security marker.

According to a fourth aspect of the present invention, there is provided a method of marking an item, the method including providing a security marker for cooperation with a detector, the detector including a molecular probe, the probe including a predetermined probe nucleotide sequence, the security marker comprising a target oligonucleotide, the target oligonucleotide comprising a pair of primer regions and a marker region located between the primer regions, the marker region comprising a predetermined marker nucleotide sequence which is fully or partially complementary to the probe nucleotide sequence.

According to a fifth aspect of the present invention, there is provided a method of detecting a marked item, the method including providing a detector for cooperation with a security marker, the detector including a molecular probe, the probe including a predetermined probe nucleotide sequence, the security marker comprising a target oligonucleotide, the target oligonucleotide comprising a pair of primer regions and a marker region located between the primer regions, the marker region comprising a predetermined marker nucleotide sequence which is fully or partially complementary to the probe nucleotide sequence.

According to a sixth aspect of the present invention, there is provided of a method of linking data to an item, the method including providing a security arrangement as described above.

Possibly, the security marker, the detector and the security arrangement include any of the features described in any of the preceding statements or following description. Possibly, the methods include any of the steps described in any of the preceding statements or following description.

A security marker may be applied by any suitable dispersion device. As an improvement to previous devices the inventors have designed a stand-alone, self-powering dispersion unit that uses a number of controlled, timed, chemical reactions to develop a slow and precise build-up of pressure within a chamber. This forces the chemical/DNA solution through a specifically designed nozzle to produce droplets over a specific time and area. The chemical reaction is triggered by a series of sensors that detect movement within the protected area and dispersion range.

Methods of detecting the security markers, according to the invention, include the following steps:

(i) Obtain photographic evidence of the area of dispersion, via monitoring by suitable movement sensors. The sensors control the dispersion and actuate an image capture device that captures evidential information to identify and locate the dispersed solution. The unique code of the solution, time and date are embedded and watermarked on the images.

(ii) Obtain a sample from an object that has potentially been labelled with a security marker, according to the invention.

(iii) Carry out a chemical reaction to identify the presence of the unique oligonucleotide code.

(iv) Amplify the marker using PCR primers that are capable of selectively binding to the marker nucleic acid.

(v) Identify the oligonucleotide sequence by a molecular technique. The technique may include any methods known in the art, such as dideoxy sequencing, mass-spectrometry sequencing, real time PCR with molecular probes or melting curve analysis.

A further aspect of the invention provides a database comprising details of where a security marker, according to the invention, is in use and a list of the oligonucleotide sequences that encode the marker within the security device. This enables the source of a security marker, according to the invention, to be identified.

The target oligonucleotide(s) may be formed synthetically.

The security marker could comprise at least two oligonucleotides, and possibly no more than 36.

The oligonucleotides may be detected and identified using polymerase chain reaction (PCR).

Each oligonucleotide may comprise a number of nucleic acid molecules, a first primer region substantially identical to a first primer; and linked to the first primer region, a marker region comprising a predetermined nucleic acid sequence capable of identifying the source of the security marker; and a region that is substantially the reverse complement of a second primer and capable of being bound by this primer.

The primer region pair may comprise the first primer region and the second primer region.

The said nucleic molecules within the marker region are designed to alter the normal complementary binding of a probe structure to allow detection at different Tms i.e. contain a fully complementary probe binding region, a single mismatch with the probe or multiple mismatches, to provide a number of identifiable DNA codes that can be used.

The target oligonucleotide(s) may be synthesized within a plasmid.

The target oligonucleotide(s) may be amplified by PCR. The target oligonucleotide(s) may be detected via melt curves (1 4) to simplify the analysis data and increase reliability.

The target oligonucleotides may comprise a pool, and may be used to create the security code when selected from the pool and assembled or mixed together.

A security assay may be performed which will analyse samples of the security marker which comprise any number of the target oligonucleotides assembled from the pool.

The molecular probes may simultaneously detect target oligonucleotides that are fully complementary to the probes, comprise a single nucleotide mismatch and comprise multiple nucleotide mismatches.

Example of typical combinations of available:

Four oligonucleotide targets per mixture=58,905 combinations

Five oligonucleotide targets per mixture=376,992 combinations

Six oligonucleotide targets per mixture=1,947,792 combinations

Seven oligonucleotide targets per mixture=8,347,680 combinations

Possibly, the nucleotide sequence of the first primer and the nucleotide sequence of the second primer are selected so that there is a low probability that their respective primer binding regions occur within 150 bases of each other in native DNA. The probability may be less than 5%. Possibly, the native DNA is from humans, dog, cat, mouse, rat, insects or prokaryotic organisms.

Possibly, the first primer region is different to the second primer region.

Possibly, the marker region comprises between 10 and 30 nucleotides.

Possibly, each primer region comprises between 15 and 50 nucleotides.

Possibly, each primer region is separated from the marker region by a stretch of 20 to 50 nucleotides.

The invention comprises use of the security marker described above to mark items, which may comprise any from the group containing: human beings, animals, liquids, materials, paper, compounds, plastics, rubber, ink, oil, perfume, polymers, grease, wax, seals, varnish, animals, items requiring a proof of authenticity, antiques, works of art, currency, objects of value.

The invention comprises a method of marking an item comprising applying the security marker as described above to the item. Possibly, the method comprises applying the security marker by means of dispersing the security marker into and over a predetermined receiving space. The item may be a person and may be a suspect involved in a crime committed in the receiving space.

The invention comprises a method of detecting the security marker described above comprising: release security marker into a receiving space; photograph the receiving space; identify a suspect person from images captured, including suspect s position and clothing during release; obtain samples from items in the receiving space which correlate to the images captured; test samples.

The sample testing may include an immunoassay test to the sample and monitor the resulting compound.

The receiving space may be scanned with ultra violet light.

Fragrance detectors may be used to detect a unique fragrance and may comprise specialized equipment or trained detection sniffer dogs.

The testing may include a step of amplifying the oligonucleotides of the security marker prior to sequencing the marker region(s). Possibly, the security marker is screened with a molecular probe, such as a HyBeacon® or TaqMan® probe. Possibly, the marker regions are sequenced by dideoxy sequencing, or mass spectrometry sequencing. Possibly, the sequence of the marker region is compared with a database to determine the source of the security marker.

The invention may provide a security kit comprising the security marker described above.

Possibly, the security marker is provided within a solution for dispersion or marking.

The invention may provide a security device, which may comprise an actuator for actuating the release of the security marker described above onto an intruder, and may include a Read Only Memory for recording the date and/or time of the release of the security marker onto the intruder and possibly for recording image files. Possibly, the security device comprises a high definition camera and/or an audio recorder which may generate still, video and/or audio files. Possibly, the still, video and/or audio files include the security code of the security marker, which may be embedded therein.

Possibly, the security device comprises monitored input and outputs complete with anti-mask and 24 hr tamper protection and internal power source.

The database may comprise data relating to any of: location; security code; ownership history; event history; product history.

### FIGURES

An embodiment of the present invention will now be described, by way of example only, and with reference to the accompanying drawings, in which:—

FIGS. 1A, 1B, 1C and 1D are melting curves for PCR-amplified target oligonucleotides using a FAM labelled HyBeacon® probe;

FIGS. 2A, 2B, 2C and 2D are melting curves for PCR-amplified target oligonucleotides using a JOE labelled HyBeacon® probe; and

FIGS. 3A, 3B, 3C and 3D are melting curves for PCR-amplified target oligonucleotides using a TAMRA labelled HyBeacon® probe.

### DESCRIPTION

The technological development has progressed down the route of generating a pool of oligonucleotides and then selecting a combination of oligonucleotides from the pool that are mixed to create a unique code and can be detected using a test method that is capable of analysing and distinguishing between multiple oligonucleotides. The output from the test method will be a unique code for each sample, which can be linked back to a management and registration database.

Oligonucleotide Pool Assembly Approach

This method was developed to reduce the cost of the device as the synthesis of oligonucleotides can be performed on a large scale to give highly pure DNA. Oligonucleotides can be tagged with fluorophores to illuminate the DNA under different wavelengths of light and hydrophobic groups can be added to improve skin or cloth binding.

The idea was to mix two to four oligonucleotides with only one base difference between them. Sequence analysis will give two peaks at the position where a nucleotide base differs between the oligonucleotides. The peaks will be at a ratio dependent on the ratio of the oligonucleotides. For example, if one of the four oligonucleotides differs from the rest at base number 5, this will show in a sequencing trace as two overlaid peaks at a ratio of 3:1, at position number 5. The pattern will be unique when oligonucleotides are mixed in different combinations.

To test this approach, eight oligonucleotides were synthesized with an 18 nucleotide primer region at the 5 and 3 ends, a 24 nucleotide central region and a 5 thiohexyl group. The central region was separated from the primer regions by two nucleotide spacers of six bases. The thiohexyl group helps with purification and acts as a sticky end to enhance binding of the oligonucleotide to the human body or fabric via sulphide-bridge formation with cysteine. The spacers help the DNA sequencing analysis due to read limitation at the start of the sequencing reaction.

A PCR assay was developed and the oligonucleotides were sequenced. However, the accuracy is highly dependent on the quality of the sequencing analysis. The short length of the oligonucleotides makes sequence analysis unreliable. To overcome the sequence analysis issues, a real time PCR assay and melting curve analysis was developed. This uses HyBeacon® probes to detect the differences between the oligonucleotides. A melt curve analysis can be used to distinguish between fully matched probes and oligonucleotides and probes that have one or more mismatches. Mismatched hybridisations melt at a lower temperature than fully matched probes and oligonucleotides. This gives a melting peak at a different position (Tm).

This method can be modified from single molecular probe to multiple molecular probes and multiple targets (multiplex PCR system). Three sets of oligonucleotides were developed, in which each comprised of one DNA target, two primers and one molecular probe. They were designed using SciTools OligoAnalyzer 3.1 from Integrated DNA Technologies.

These oligonucleotides have been designed with no significant hairpin loop formations within the target and no significant dimers between oligonucleotides. They are also not found in human, animal or plants that have been recorded in the NCBI database. Thus false positive results from contamination should not occur. The benefit of this method is that the Tms of the hybridized molecular probe and oligonucleotide will yield a unique pattern in a melt curve analysis. Calculated Tms of design 1, 2 and 3 are 59, 65 and 69° C. (calculated at 3 mM Mg and 0.5 pM dNTP) and expected Tms are 56, 62 and 66° C., respectively.

The assays have been tested under the following PCR conditions; 20 pL reaction volumes with 0.5 μM of reverse primer (RP), 0.05 pM of forward primer (FP), 10 ng/pL BSA, 75 nM of probe, 0.5 mM dNTP, 3.0 mM MgCl₂, 0.5 units of HotStar Tag polymerase (Qjagen, UK) and 2 ng total quantity of oligonucleotide. Cycling protocols were activation of the enzyme at 95° C./15 min, 35 cycles of 94° C./15 s, 50° C./20 s, 72° C./30 s. The Tms of the probes (H1, H2 and H3) and oligonucleotide (T1, T2, and T3) duplexes were measured after PCR amplification by heating to 95° C. at 20° C./s, cooling to 30° C. at 0.5° C./s, hold at 30° C. for 2 min, prior to performing a melting curve analysis to 80° C. at 0.1 ° C./s in continuous fluorescence requisition mode. The melting peaks were generated from the first derivative, −dF/dT, from the melting curve (F/T).

Different experiments were performed, including single oligonucleotide/single probe, multiple oligonucleotides/single probe and multiple oligonucleotides/multiple probes, using real time PCR and melting curve analysis. The Tms of the single probe/single oligonucleotide of design 1, 2 and 3 were 55.9, 59.4 and 63.5° C., respectively. The Tms of the single molecular probe/mixed oligonucleotides (all three oligonucleotides) were the same as when using a single oligonucleotide. This indicates that the presence of other DNA will not interfere with the probe binding to a specific oligonucleotide. The Tms of multiple probes /mixed oligonucleotides gave two melt curves, which indicates that the assay could be used for multiplex PCR and melting curve analysis. This assay can be used to identify different DNA, using separate probe (three PCR tubes) detection or single tube analysis (multiplex PCR).

The advantage over the previous development is that the oligonucleotide batches can be mass produced in advance to reduce the manufacturing cost. The technology can also be scaled up as required.

This method has also been tested using three TaqMan® probes labeled with FAM, JOE and TAMRA fluorescent dyes. Real time PCR has been used to show that these can successfully detect three matched oligonucleotides, over a serial dilution, in a multiplex reaction that contains all three probes, as shown below. However, melting curve analysis cannot be performed with these probes as they are irreversibly hydrolyzed during the PCR reaction to separate the fluorescent dye and quencher.

The inventors realized that adding one or more mismatches between the oligonucleotides and the molecular probes provides an additional permutation factor. The degree of mismatch or complementarity can be identified in a melting curve and matched to a database. This design has been taken forwards to the final assay, as discussed below.

Final Design

A pool of 36 target oligonucleotides has been designed. Each target oligonucleotide has primer binding sites for one of a set of four primer pairs at the ends of the oligonucleotide. The central region of the oligonucleotide is complementary to one of three probes. The central region is either fully complementary (ie has no nucleotide mismatches), has one nucleotide mismatch or has two or more mismatches.

Each target oligonucleotide comprises a target identity, the target identity comprising a probe identity relating to the respective complementary probe, a primer identity relating to the respective primer region pair and a complementarity identity relating to the number of nucleotide mismatches between the respective complementary probe nucleotide sequence and the marker nucleotide sequence.

In one example, one of the oligonucleotide target identities is V5T1A.

V5 comprises the primer identity. Other values could be V15, V18 and V19.

T1 comprises the probe identity. Other values could be T2 and T3.

A comprises the complementarity identity and indicates that the probe is fully complementary to the target oligonucleotide. Other values could be B, indicating one mismatch and C, indicating two or more mismatches.

The oligonucleotides have been synthesized and purified by well-known and documented standard methods. A pool of oligonucleotides will be made to give 106 copies per ml. The pool will be mixed with 50% ethanol and an ultraviolet compound. The ethanol provides stability and increases the speed of drying once the mix has been applied to a surface. The UV compound gives an identifiable area on which the oligonucleotides will be found and aids recovery. The mix will be added to the security device and used as required. As the number of oligonucleotides included in the device increases, the number of possible combinations also increases. For example, if four oligonucleotides are used, 58,905 combinations are possible. However, this is reduced slightly when pool design takes into account rules that allow for easier analysis (see later section).

A PCR analysis has been developed using four pairs of primers. Each primer pair is used in a separate reaction and the oligonucleotide pool was tested with each of the four reactions. Each of the reactions also contains three probes. The probes are labeled with either FAM, JOE or TAMRA fluorescent dye. The probes hybridize to the three different central regions of the oligonucleotides. After the PCR reaction, a melt curve analysis is performed where the temperature is ramped up from 30° C. to 80° C. and fluorescence is monitored continuously. The probes have a greater fluorescence when bound to their target than when in free solution. As the temperature increases and the probe dissociates from the target, fluorescence is reduced. Each probe shows a slightly different melting temperature when there is zero, one or multiple mismatches with the target. This allows for discrimination between the amplified oligonucleotides in the pool, as shown below.

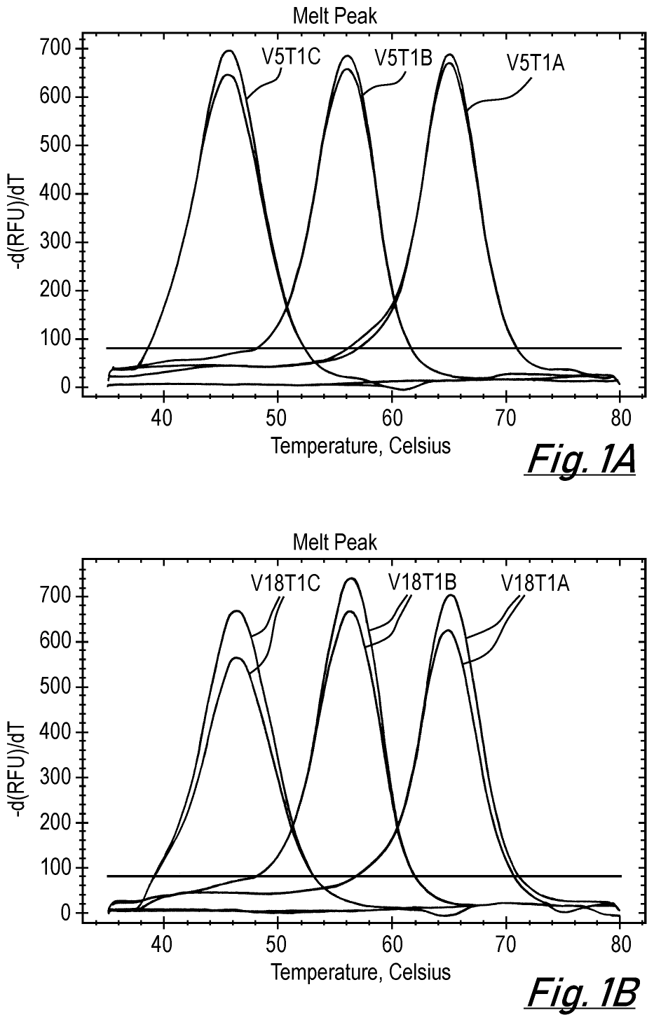

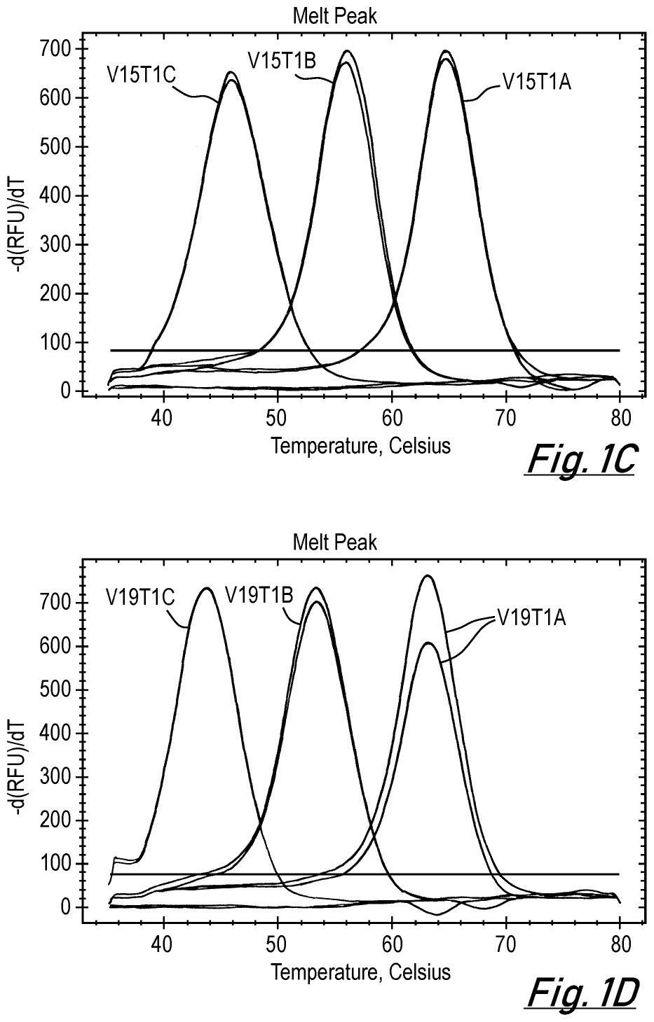

FIGS. 1A, B, C and D show detection and identification of PCR-amplified target sequences using the FAM labelled HyBeacon® probe. High quality peaks are generated with the T1A, T1Band T1C targets.

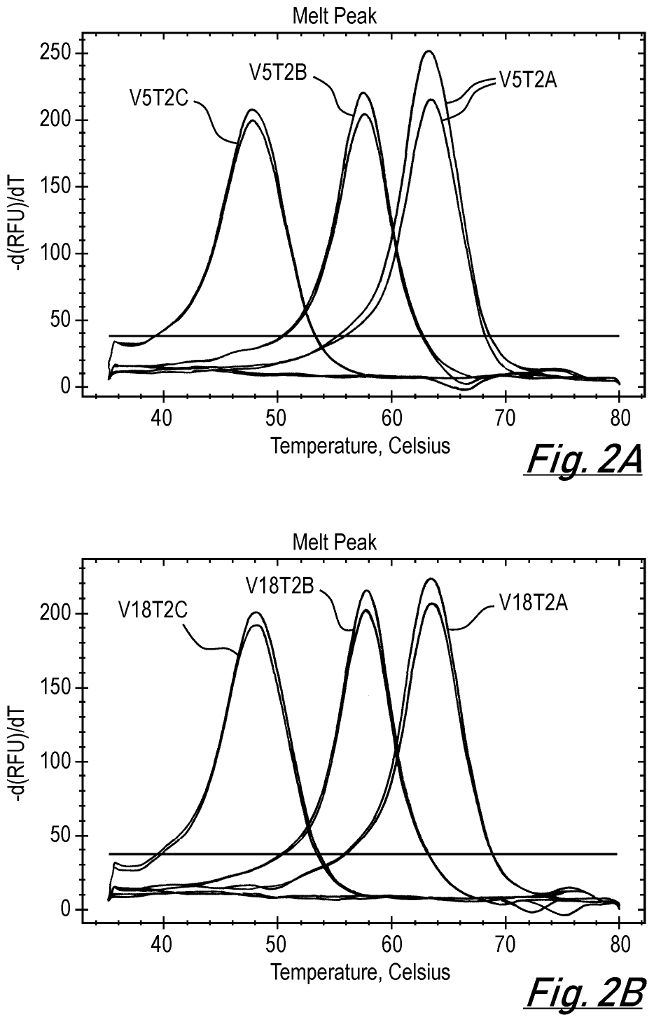

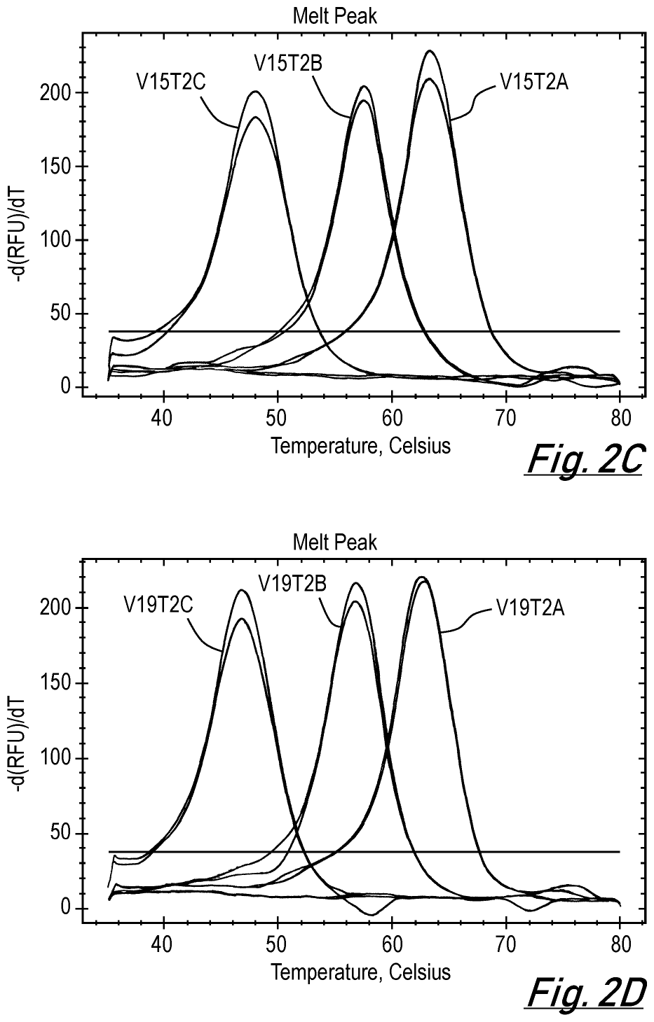

FIGS. 2A, B, C, and D show detection and identification of PCR-amplified target sequences using the JOE-labelled HyBeacon® probe. High quality peaks are generated with the T2A, T2B and T2C targets.

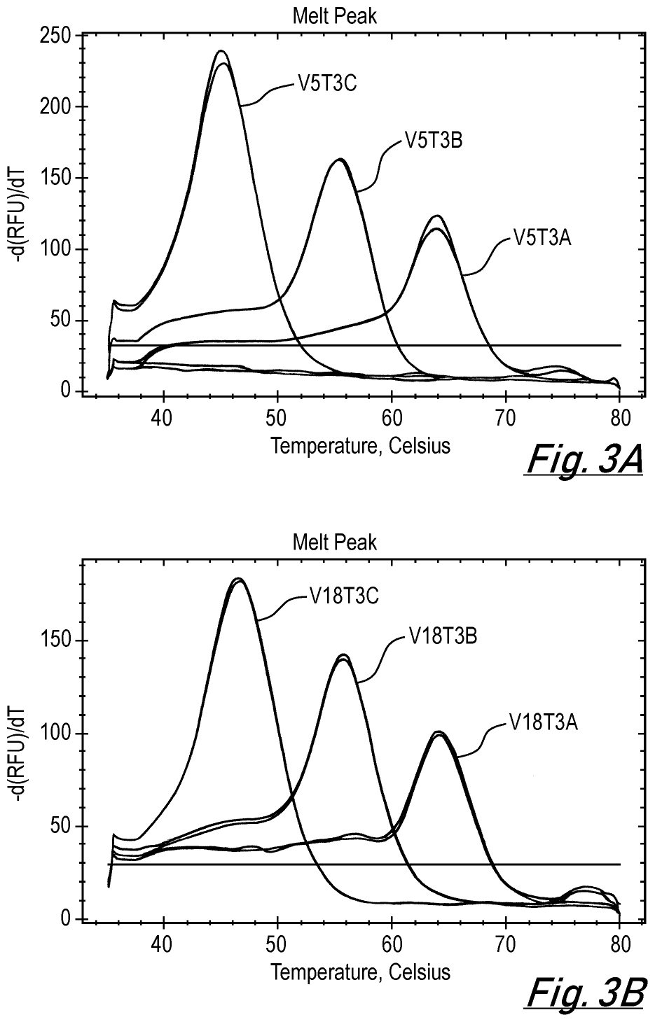

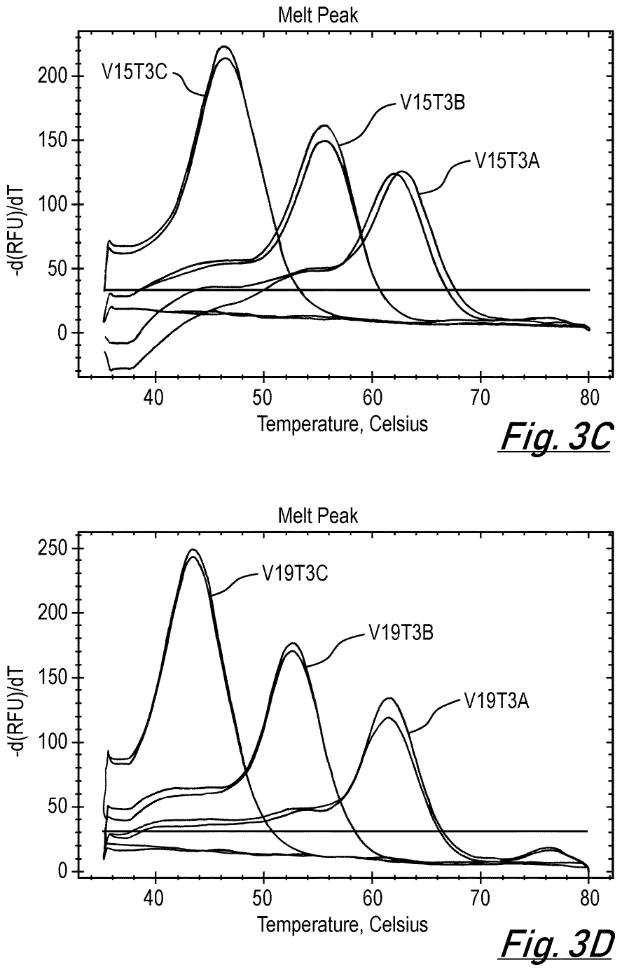

FIGS. 3A, B, C and D show detection and identification of PCR-amplified target sequences using the TAMRA-labelled HyBeacon® probe. High quality peaks are generated with the T3A, T3B and T3C targets.

Table 3 shows how this assay method allows the four PCR reactions to detect the 36 oligonucleotides as follows:

TABLE 3
Detection of oligonucleotides in the four reactions.

| Tube | Primers | Probe | Target | Probe | Target | Probe | Target |
|---|---|---|---|---|---|---|---|
| 1 | V5 | HYB- | V5T1A | HYB- | V5T2A | HYB- | V5T3A |
| | | CS1 (T1) | V5T1B | CS2 (T2) | V5T2B | CS3 (T3) | V5T3B |
| | | | V5T1C | | V5T2C | | V5T3C |
| 2 | V15 | HYB- | V15T1A | HYB- | V15T2A | HYB- | V15T3A |
| | | CS1 (T1) | V15T1B | CS2 (T2) | V15T2B | CS3 (T3) | V15T3B |
| | | | V15T1C | | V15T2C | | V15T3C |
| 3 | V18 | HYB- | V18T1A | HYB- | V18T2A | HYB- | V18T3A |
| | | CS1 (T1) | V18T1B | CS2 (T2) | V18T2B | CS3 (T3) | V18T3B |
| | | | V18T1C | | V18T2C | | V18T3C |
| 4 | V19 | HYB- | V19T1A | HYB- | V19T2A | HYB- | V19T3A |
| | | CS1 (T1) | V19T1B | CS2 (T2) | V19T2B | CS3 (T3) | V19T3B |
| | | | V19T1C | | V19T2C | | V19T3C |

To provide robust calling of the oligonucleotides present in the pool, pools would be assembled using just one oligonucleotide per probe for any of the four reactions, i.e. the fully matched, single mismatch and multiple mismatch oligonucleotides, with the same primer pair, are not used together in the same pool. Using this rule, with four oligonucleotides in a pool, the number of possible combinations is reduced from 58905 to 40095. However, the system is capable of discriminating between more oligonucleotides per probe and between more than four oligonucleotides per pool. This gives the system the capability to use a much higher number of combinations.

The four PCR reactions have been developed to run on two different instruments; the ParaDNA and the BioRad CFX. The ParaDNA instrument is portable and automated software has been developed to provide an output that consists of the oligonucleotides present in the sample. The user is not required to perform any analysis steps.

The BioRad CFX is a standard laboratory real time PCR instrument. An SOP has been produced for the running of the instrument and the user is required to follow analysis steps to output a file that can be run through a Microsoft Excel macro. This macro will output the oligonucleotides present in a sample.

The analysis from both instruments can be fed back into the database to establish the identity of the device that the sample originates from.

During analysis, the PCR cycling conditions have been optimized for both instruments. The following cycling parameters, which incorporate the PCR amplification and the melt curve, are used:

TABLE 6
Cycling Parameters for Biorad CFX

| Phase | Temperature (° C.) | Duration (s) | Cycles | Rate |
|---|---|---|---|---|
| Denature | 98 | 60 | 1 | N/A |
| PCR | 98 | 5 | 42 | N/A |
| | 65 | 10 | | |
| Heat | 98 | 60 | 1 | N/A |
| Cool | 30 | 60 | 1 | 0.2 |
| Melt | 80 | N/A | 1 | 0.5 |

TABLE 7
ParaDNA

| Phase | Temperature | Duration | Cycles | Rate |
|---|---|---|---|---|
| Denature | 98 | 60 | 1 | 7 |
| PCR | 95 | 10 | 42 | 7 |
| | 65 | 20 | | |
| Heat | 98 | 60 | 1 | 7 |
| Cool | 30 | 30 | 1 | 0.2 |
| Melt | 85 | N/A | 1 | 0.1 |

During development the reagent concentrations have been optimized. The PCR master mix consists of the following, when running the test on the BioRad CFX:

TABLE 8
Biorad CFX PCR reagents and volumes for 10 reactions. 15 μl
of this mix is used per reaction, with 5 μl of sample.

| | | Final | | Volume | |
|---|---|---|---|---|---|
| Reagent | Supplier | concentration | | μl | N = 10 |
| Phire buffer | Thermo | 1x | | 4 | 40 |
| | Scientific | | | | |
| dNTPs | Fisher | 1 | mM | 0.4 | 4 |
| | Scientific | | | | |
| BSA | Roche | 0.15 | mg/mL | 0.6 | 6 |
| Carrier RNA | QIAGEN | 0.1 | μg/μl | 2 | 20 |
| Forward | Sigma-Aldrich | 250 | nM | 0.5 | 5 |
| primer | | | | | |
| Reverse | Sigma-Aldrich | 1 | μM | 2 | 20 |
| Primer | | | | | |
| HYB-CS1 | ATDBio | 450 | nM | 0.9 | 9 |
| HYB-CS2 | ATDBio | 450 | nM | 0.9 | 9 |
| HYB-CS3 | ATDBio | 450 | nM | 0.9 | 9 |
| Water | Sigma-Aldrich | | | 2 | 20 |
| Phire | Thermo | | | 0.8 | 8 |
| Polymerase | Scientific | | | | |

TABLE 9
ParaDNA PCR reagents and volumes for 10 reactions. 25 ml
of this mix is used per reaction, with 5 ml of sample.

| | | Final | | Volume | |
|---|---|---|---|---|---|
| Reagent | Supplier | concentration | | μl | N = 10 |
| Phire buffer | Thermo | 1x | | 6 | 60 |
| | Scientific | | | | |
| dNTPs | Fisher | 1 | mM | 0.6 | 6 |
| | Scientific | | | | |
| BSA | Roche | 0.15 | mg/mL | 0.9 | 9 |
| Carrier RNA | QIAGEN | 0.08 | μg/μl | 2.4 | 24 |
| Forward primer | Sigma-Aldrich | 222 | nM | 0.67 | 6.7 |
| Reverse Primer | Sigma-Aldrich | 1 | μM | 3 | 30 |
| HYB-CS1 | ATDBio | 450 | nM | 1.35 | 13.5 |
| HYB-CS2 | ATDBio | 450 | nM | 1.35 | 13.5 |
| HYB-CS3 | ATDBio | 450 | nM | 1.35 | 13.5 |
| Water | Sigma-Aldrich | | | 0.18 | 1.8 |
| Phire | Thermo | | | 1.2 | 12 |
| Polymerase | Scientific | | | | |
| Type IX-A | Sigma-Aldrich | 1% | | 6 | 60 |
| Agarose | | | | | |

The test has been validated for both instruments. The BioRad CFX assay has been validated using 24 mixtures of three of the 36 oligonucleotides, at a total oligonucleotide concentration of 103 copies/μl. Four no template controls were also tested. The validation showed that the assay is able to accurately detect and report the oligonucleotides present in mixtures. The assay meets the following criteria:
High quality melting curves were generated with the CFX instrument.
100% of test calls were in agreement with the mixtures.
The first time pass rate for the 24 mixtures was 100%. None of the samples needed to be retested.
Melting peaks were not generated with any of the no template control samples.

The validation on the ParaDNA instrument was performed with 12 mixtures of oligonucleotides, which contained two to four oligonucleotides, and two no template controls. The oligonucleotide calling software was used to analyze the data and provide the assignment of the codes. All 12 samples were called correctly, with no false positives, false negatives or incorrectly assigned calls.

Oligonucleotide Recovery

The oligonucleotide pool will be recovered from a substrate, after activation of the device, by standard forensic procedures, e.g. cotton or rayon swabs. Recovery has been investigated using both the ParaDNA and Biorad CFX tests, from pig skin, a glass microscope slide and a fabric sample (cotton). Three methods of recovery were used; direct sampling, indirect sampling and expressed sampling. Direct and indirect sampling were both tested with the ParaDNA assay. Direct sampling involved the use of a specific sampling tool designed for use with the ParaDNA instrument. The tip of the tool is used to collect the sample by contact with the substrate and the tool head is then transferred to the four PCR reaction tubes. Indirect sampling is the use of a moistened cotton swab to sample the substrate. The tip of the swab is then sampled with the ParaDNA sampling tool. Expressed sampling is when the head of the swab is placed in a micro centrifuge tube containing 500 μl of tissue culture water. This is then vortexed to remove the oligonucleotides from the cotton swab and the resultant solution is used in the PCR reaction.

Alternatively, expressed sampling can also be performed with excised fabric rather than the head of the cotton swab.

Recovery was assessed in a sensitivity study, using the different recovery methods. Firstly, different mixtures of oligonucleotides at 50'10³, 10⁴, 10⁵, 10⁶ and 10⁷ copies per pl were tested. The minimum levels of oligonucleotide that gave correct calls were 50×10⁶ and 50×10⁷. These levels were confirmed as acceptable concentrations of oligonucleotides for both the ParaDNA and Biorad CFX assays. The expressed method of recovery proved to be the best method for the Biorad CFX assay, whilst the direct method was best for the ParaDNA assay. However, for the skin sample, the indirect method was preferred for the ParaDNA assay. The excised fabric method may also improve the method for fabric. The 50×10⁶ level of oligonucleotide was also tested with the oligonucleotides synthesized into a plasmid. This provides a longer DNA target for the assay and reduces the need for dilution, quantification and equalization of oligonucleotides. Plasmids may offer a slight increase in sensitivity but will be synthesized at a lower concentration and, therefore, will increase the cost.

Other recovery methods have been tested during development, such as swabbing using ethanol and skin scrapes. Dithiothreitol (DTT) has been found to be useful in isolating security markers when phosphothioate is incorporated into the oligonucleotide. The DTT denatures any disulphide bonds between the phosphothioate and proteins on the object that has been marked, thus releasing the security marker. Hapten or a number of different haptens may also be used for different situations. For example, a specific hapten may be used in one country and not another, thus allowing the identification of crime suspects when they pass through, for example, customs. Alternatively, different haptens may be used for different situations; for example, to demonstrate whether a person has carried out a car theft or has been involved in breaking and entering a factory premises.

In addition, the markers presence can be uniquely identifiable by attachments made or mixed with the oligonucleotides using overt or covert materials, such as ultra-violet compounds with a spectrum of colours or a library of unique fragrances.

The inventors have also designed a method of identification using a small molecule which, when combined with a larger carrier, such as a protein, promotes a change of state (contrast) that can be visually detected and extracted for analysis. This method can include antibodies or any other system that allows for a measureable state change.

Stability

Studies have been performed to check the stability of oligonucleotides over 18 months in storage buffer solution, at room temperature, using two strategies:

Capillary electrophoresis

(ii) PCR at estimated concentrations

Capillary electrophoresis (CE) is extremely high resolution and separates DNA strands of similar length. It gives more reliable purity data for oligonucleotides than normal reversed-phase HPLC (High-performance liquid chromatography), ion exchange, HPLC or conventional gel electrophoresis. The separation is based on the size to charge ratio and uses a small capillary filled with electrolyte. The longer length oligonucleotides move more slowly than shorter oligonucleotides. Thus, degradation of the oligonucleotides can be followed by this method.

Eighteen samples of 0.5 OD of oligonucleotide res0170 and six samples of res0173 were prepared in buffer (1.0 mL, 20 mM sodium phosphate buffer pH 7.8, 100 mM NaCl), under sterile conditions and kept in sterile plastic tubes (2 mL, self-standing, Simport, Canada), in the dark at room temperature. The res0170 oligonucleotide was used for monthly stability testing, whilst res0173 was tested every three months.

Analysis was carried out using a Beckman Coulter P/ACE MDQ. Capillary Electrophoresis system, with 32 Karat software and UV monitoring at 254 nm. A solution of an oligonucleotide in 100 μL of water was injected into the gel for 5-20 sec, with an injection voltage of 10.0 KV and a separation voltage of 9.0 KV. 100-R Gel was used with Tris-borate buffer and 7 M urea (Kit No 477480).

To date, the stability of res0170 and res0173 has been measured up to month three. The purity of the DNA has been good, showing only one peak. After three months of storage no degradation peaks have been observed.

PCR at estimated concentrations experiments used a 5 pg sample in cloth/skin to calculate the estimated concentration of the DNA solution. Previous experiments have shown that the smallest quantity of DNA that can be detected without giving false positive results is 0.5 pg.

However, a higher amount will give better results and be easier to detect. Two oligonucleotides, res0171 and res0172 have been used, with a molecular weight of approximately 22100Da.

We assumed that one drop of 0.5 μL was on the cloth or skin after spraying and the percentage DNA recovery was 60% (0.3 μL contain 5 μg). Thus, we used 8.3 pg in a 0.5 μL solution. This equates to 733 pmolar (pM) (approximately 16 μg/Litre). A 50 μL volume of 733 pM DNA solution (10 mM sodium phosphate buffer, 100 mM NaCl, pH 7.8) was aliquoted into sterile tubes (20 tubes of each oligonucleotide) and kept in the dark at room temperature. A PCR assay was performed every month and analysed using agarose gel electrophoresis. The gel analysis of the PCR assays, over zero, one and two months shows only one product. Thus, it can be concluded that at a concentration of 16 μg/L, there is no degradation of the oligonucleotide.

In Use

In use, a security marker is assembled which has a unique combination of the target oligonucleotides from the pool and thus comprises a security code, which comprises the target identity or identities of the or all of the target oligonucleotides. The security code can be stored in a database and can relate to data which can then be retrieved by the analysis described.

Advantageously, when applied to a suspect, the security marker is virtually impossible to remove, since the oligonucleotides bind to skin and clothing. For the same reason the security marker of the invention can be used for a variety of other uses, such as providing identification and authenticity for a wide variety of items such as art objects; currency notes; high value items; antiques and by a variety of different application means.

In addition to using the marker to identify a person or object, linking them to a place and time, coupled with supporting evidential information, such as via ink within a pen or writing device; an ink cartridge within a pen or writing device; a UV pen or security marker; an ink printer cartridge; printing material; printed material; paper; plastic; rubber; paint; seal wax or seal material; sealer or protective varnish; glue; paste; fabric; water; and in fact any liquid that requires identification and authenticity proof of ownership.

The data in the database could include a history of the item to provide provenance.

Final Remarks

The present invention has been found to have the following advantages:

It provides a unique marker with the advantages of DNA code sensitivity and robust nature, whilst increasing production efficiency to reduce costs and increase the applications.

It provides a unique marker that can be analysed quickly and efficiently using portable equipment, or in the laboratory, without any prior knowledge of the sample to be tested (essential for use in the security sector).

It provides a unique marker that can be produced in volume, stored and assembled as required. It provides a unique marker that can be recovered and identified using simple techniques already used and approved by law enforcement officers.

It provides a unique marker that can be analysed to reliably and efficiently provide identification data.

1. A security marker for cooperation with a detector, the detector including a molecular probe, the probe including a predetermined probe nucleotide sequence, the security marker comprising a target oligonucleotide, the target oligonucleotide comprising a pair of primer regions and a marker region located between the primer regions, the marker region comprising a predetermined marker nucleotide sequence which is fully or partially complementary to the probe nucleotide sequence.

2. A security marker according to claim 1, in which the marker region is selected from the group containing:
the marker region is fully complementary to the probe nucleotide sequence, the marker nucleotide sequence including no nucleotide mismatches with the probe nucleotide sequence,
the marker region is partially complementary to the probe, the marker nucleotide sequence including a single nucleotide mismatch with the probe nucleotide sequence,
the marker region is partially complementary to the probe, the marker nucleotide sequence including two or more nucleotide mismatches with the probe nucleotide sequence.

3. A security marker according to claim 1, in which the security marker includes a plurality of the target oligonucleotides, each of which is different from the others.

4. A security marker according to claim 3, in which the detector includes a plurality of the probes, the security marker includes a corresponding plurality of probe sets of the target oligonucleotides, each probe set corresponding to a different probe so that the target oligonucleotides of each probe set are fully or partially complementary only to the respective corresponding probe.

5. A security marker according to claim 3, in which the security marker includes a plurality of primer region sets, each set comprising target oligonucleotides having the same pairs of primer regions, and the primer region pairs of the target oligonucleotides of each primer region set are different to the primer region pairs of the target oligonucleotides of the other set(s).

6. A security marker according to claim 1, in which the or each target oligonucleotide comprises a target identity, the target identity comprising a probe identity relating to the respective complementary probe, a primer identity relating to the respective primer region pair and a complementarity identity relating to the number of nucleotide mismatches between the respective complementary probe nucleotide sequence and the marker nucleotide sequence, the security marker comprising a security code which comprises the target identity or identities of the or all of the target oligonucleotides.

7. A security marker according to claim 1, in which the target oligonucleotide(s) comprising the security marker is/are selected from a pool comprising a plurality of the target oligonucleotides, and in which each of the target identities of the target oligonucleotides in the pool are different from each other.

8. A security marker according to claim 1, in which the or each target oligonucleotide is identified by subjecting the security marker to one PCR reaction for the or each primer pair in the presence of the or all of the probes, performing a melt curve analysis to generate melting temperature curves for the or each of the primer pairs, and then analysing the curves to generate the security code.

9. A detector for cooperation with the security marker of claim 1, the detector the detector including a molecular probe, the probe including a predetermined probe nucleotide sequence, the security marker comprising a target oligonucleotide, the target oligonucleotide comprising a pair of primer regions and a marker region located between the primer regions, the marker region comprising a predetermined marker nucleotide sequence which is fully or partially complementary to the probe nucleotide sequence.

10. A detector according to claim 9, in which the detector includes a plurality of the molecular probes, each probe having a different probe nucleotide sequence.

11. A detector according to claim 9 in which the molecular probe comprises a hybridisation beacon, which comprises a fluorescent material.

12. A security arrangement including the security marker of claim 1, in which the arrangement includes a device for applying the security marker to an item, and a database which holds data relating to the security marker.

13. A security arrangement according to claim 12, including the detector for detecting the security marker.

14. A method of marking an item, the method including providing a security marker for cooperation with a detector, the detector including a molecular probe, the probe including a predetermined probe nucleotide sequence, the security marker comprising a target oligonucleotide, the target oligonucleotide comprising a pair of primer regions and a marker region located between the primer regions, the marker region comprising a predetermined marker nucleotide sequence which is fully or partially complementary to the probe nucleotide sequence.

15. A method according to claim 14, wherein each target oligonucleotide is identified by subjecting the security marker to one PCR reaction for the or each primer pair in the presence of the or all of the probes, performing a melt curve analysis to generate melting temperature curves for the or each of the primer pairs, and then analysing the curves to generate the security code.

16. A method of detecting a marked item, the method including providing a detector for cooperation with a security marker, the detector including a molecular probe, the probe including a predetermined probe nucleotide sequence, the security marker comprising a target oligonucleotide, the target oligonucleotide comprising a pair of primer regions and a marker region located between the primer regions, the marker region comprising a predetermined marker nucleotide sequence which is fully or partially complementary to the probe nucleotide sequence.

17. A method according to claim 16, in which the detector includes a molecular probe comprising a hybridisation beacon, which comprises a fluorescent material.

18. A method of linking data to an item, the method including providing a security arrangement according to claim 12.
